(19) United States
(12) Patent Application Publication
(10) Pub. No.: US 2005/0209625 A1
(43) Pub. Date: Sep. 22, 2005

(54) Method and apparatus for electrical stimulation to enhance lancing device performance

(76) Inventor: Frank A. Chan, Sunnyvale, CA (US)

Correspondence Address: Woodard, Emhardt, Moriarty, McNett & Henry LLP;Bank One Center/Tower, Suite 3700, 111 Monument Circle, Indianapolis, IN, 46204-5137, US

(21) Appl. No.: 10/791,173

(22) Filed: Mar. 2, 2004

(51) Int. Cl. A61B 17/14

(52) U.S. Cl. 606/181

(57) ABSTRACT

A lancing device where electrical stimulation is applied to a skin sampling site prior to making an incision to achieve at least one of pain masking and bodily fluid engorgement at the site. The electrical stimulation is supplied by a low current high voltage AC signal generator in sufficient quantities to produce vasodialation and/or pain masking.

### FIELD OF THE INVENTION

The present invention relates to devices and methods for obtaining samples of blood and other fluids from the body for analysis or processing.

### BACKGROUND OF THE INVENTION

The wide-spread application of devices for extracting samples of bodily fluids for analysis such as determining blood glucose level has led to significant activity in the field to address several problems and issues. These are the problems of pain when the skin is pierced by a lance and the problem of insuring a sufficient quantity of blood at the surface to obtain a proper sample size. Many proposals have been made to achieve these ends.

In terms of pain management there are there have been developments relative to the shape of the lance itself. These have involved depth of cuts so that the depth is the minimum necessary to extract a sample. In addition, the rate of incision has been controlled so that with a faster incision, pain is diminished.

Other activities have focused on pain masking by using vibrators and even patient distractions so that a patient is not focusing on the pain that will be experienced during the process.

A second area of effort focuses on stimulating increased presence of blood so that at least a minimum blood sample size is collected after lancing for accurate testing. Some research has focused on ways of palpating the skin to express additional blood, either manually or by various mechanisms. Other researchers have proposed using vibration, ultrasonics and other stimulation to increase blood flow. However, such devices are either too crude and simplistic or are overly complicated and expensive.

The above activity is brought into increased focus when alternate site testing (AST) is adopted to sample bodily fluids from locations other than the fingers. Both pain minimization and blood engorgement need to be managed.

### SUMMARY

The invention, in one form, relates to a device for obtaining a sample of bodily fluid through the skin. The device comprises a housing and electrodes on the housing positioned to contact a site on the skin. An electrical signal generator applies electrical energy to the electrodes in sufficient quantity to stimulate the skin at the site to accomplish at least one of pain masking and bodily fluid engorgement at the site. A skin-lancing device mounted in the housing directs a skin-lancing medium against the skin at the site to form an incision therein subsequent to the application of electrical energy.

In another form, the invention relates to a method obtaining a sample of bodily fluid through the skin. The method comprises applying electrical energy to a sampling site on the skin of sufficient quantity to stimulate the skin at the site to accomplish at least one of pain masking and bodily fluid engorgement at the site. Subsequently, an incision is formed at the site to remove a sample of bodily fluid.

### BRIEF DESCRIPTION OF THE DRAWINGS

FIG. 1 shows a highly schematic drawing showing the application of the present invention to a lancing device.

FIG. 2 shows a perspective view of one set of electrodes and skin contacting end wall configuration for use in the lancing device of FIG. 1.

FIG. 3 shows an alternative array of electrodes and end wall design for the lancing device of FIG. 1.

FIG. 4 shows a simplified circuit diagram for the signal generator shown schematically in FIG. 1.

### DESCRIPTION OF THE SELECTED EMBODIMENT

For the purposes of promoting an understanding of the principles of the invention, reference will now be made to the embodiments illustrated herein and specific language will be used to describe the same. It will nevertheless be understood that no limitation of the scope of the invention is thereby intended. Any alterations and further modifications in the described processes, systems or devices, and any further applications of the principles of the invention as described herein, are contemplated as would normally occur to one skilled in the art to which the invention relates.

The present invention uses electrical treatment of a skin sampling site to achieve one or both of pain masking and blood engorgement before a lancing device causes an incision to be made for blood sampling.

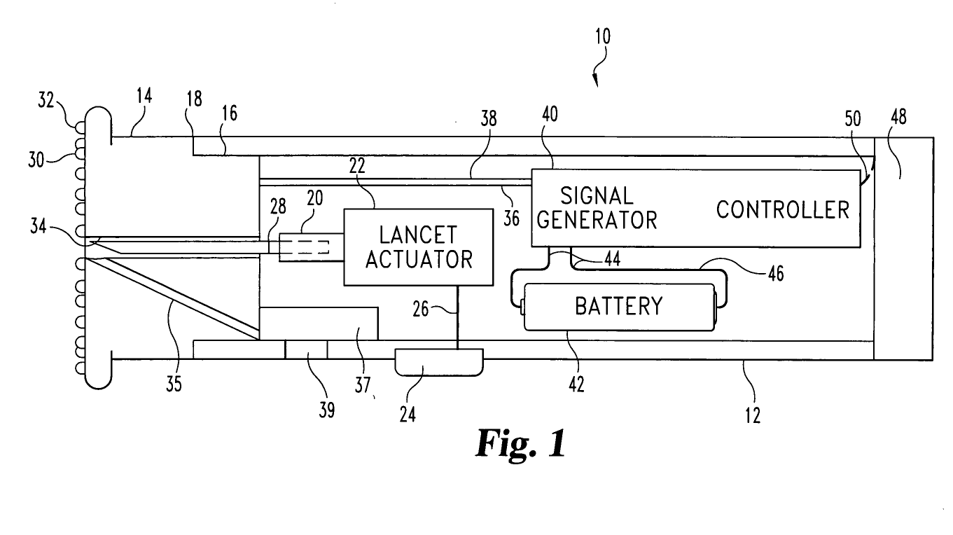

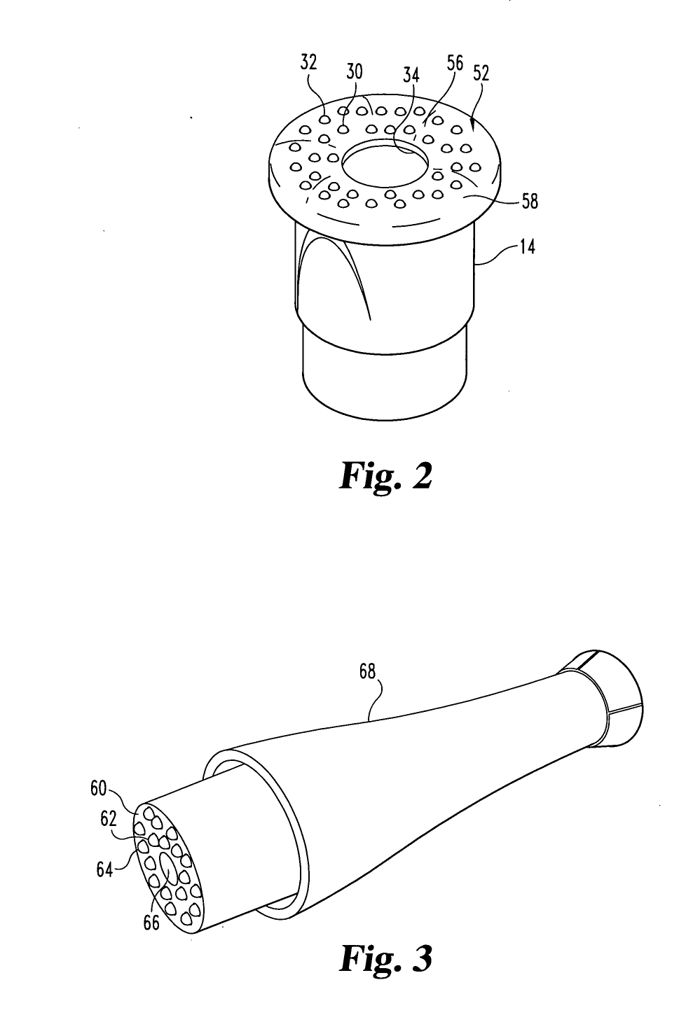

Referring to FIG. 1, there is shown a lancing device 10 comprising a housing 12 which may be annular in form. Housing 12 has skin contacting end cap 14 which may take the form shown in FIG. 2 or FIG. 3 as discussed below. End cap 14 has an end section 16 removably connected to the end 18 of housing 12. Mounted within housing 12 is a lancet holder 20 connected to a lancet actuator 22. Lancet actuator 22 is responsive to an operator controlled button 24 through interconnection 26 to cause lancet carrier 22 to displace a lancet 28 to the left as viewed in FIG. 1 to pierce the skin adjacent to device 10. It is also possible to use the pressure of cap 14 against a skin site through an interconnection (not shown) with lancet actuator 22 to displace lancet 28. Lancet actuator 22 is adapted to have a controlled rate of displacement and depth of penetration to provide optimum withdrawal of bodily fluid such as blood. Lancet 28 is removable so that it may be disposed in appropriate fashion after a test is completed. Although making a mechanical incision is described for piercing the skin, it should be apparent that other mechanisms for making an incision, such as a laser, could be used with the present invention.

Lancet actuator 22 may take one of many different forms to achieve a controlled rate of displacement and penetration depth for the lancet 28. Lancet actuator 22 may be mechanical in form using a spring-like device. It may also be electrically or pneumatically actuated. As herein shown, a capillary passage 35 leads from the mouth of passage 34 adjacent the incision of lancet 28 to a sensor 37 which gives an indication of bodily fluid parameter or condition through optical read-out 39. Alternatively, lancet 28 may to pierce the skin so that a sufficient quantity of blood may accumulate on the skin for application to a test strip (not shown). It should be noted that to those skilled in the art, the unit may be used to collect blood samples through the lancet 28 and provide still another way to integrate the testing process.

The advantages and features of the present invention will be seen to be equally applicable to the range of devices used to sample blood for glucose measurement and other applications. More specifically, the invention would be applicable to devices that sample and analyze the blood in a single unit.

In accordance with the present invention, the lancing device cap 14 has a plurality of electrodes 30 and 32 grouped within sets. The electrodes 30 and 32 are positioned in an array around the periphery of an opening 34 for lancet 28. As described below, the cap 14 may take the form shown in FIG. 2 or in FIG. 3. The electrode sets 30 and 32 may be deployed on the head in a variety of arrays to achieve the objectives of the present invention. They may be positioned in a random fashion with pairs positioned adjacent one another without any specific orientation. Alternatively, the pairs may be arranged in circumferential fashion around the opening 34. A further orientation may be in radial arrays. Based on present experimentation, the random orientation of the electrodes allowed achievement of the objectives of the invention. It should be apparent to those skilled in the art that the electrodes may be oriented other than in the random fashion and still achieve objectives of the present invention.

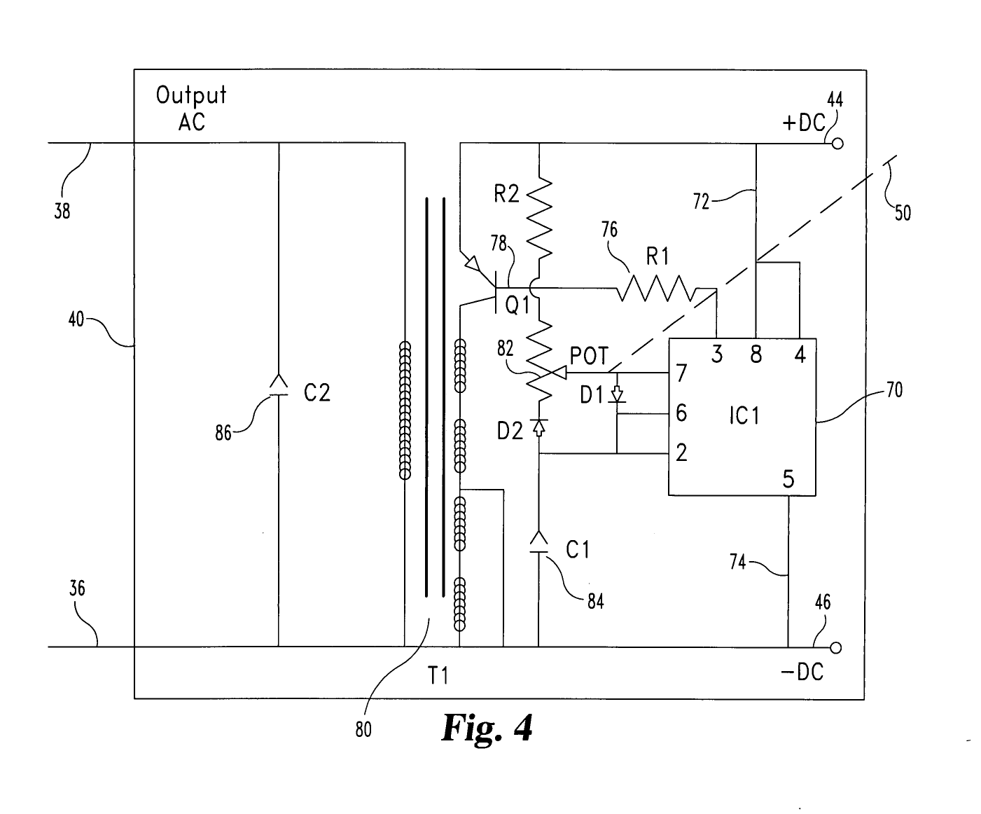

As shown in FIG. 1, the electrodes 30 and 32 are connected by lines 36 and 38 to a signal generator and controller 40. Signal generator 40 is supplied with electrical power from a power source such as a battery 42 via lines 44 and 46. As shown in FIG. 4, signal generator 40 comprises an integrated circuit (IC) oscillator 70 having input leads 72 and 74. Oscillator 70 provides an output on terminal 3 via resistor 76 to the gate of a transistor 78. Transistor 78 is connected between line 44 and 46 on the input to a step up transformer 80. Output terminal 7 of oscillator 70 provides an input to a variable resistor 82 so as to control the frequency of oscillator 70. The output side of transformer 80 is connected to output leads 36 and 38 which lead to the electrodes 30 and 32. Capacitors 84 and 86 provide smoothing of the output wave. The transistor 78 acts to pass current through the input side of transformer 80 in approximately a square wave. The transformer 80 increases the voltage output to an equivalent square wave on the output side. Capacitor 86 smoothes the wave form so that it ends up being a high voltage AC waveform. Variable resistor 82 is adjustable by means of an operator-manipulated knob 48 via an appropriate connection indicated by dashed line 50.

Signal generator 40, as illustrated, is of a type that generates a high voltage AC wave. The voltage level can be approximately from 10 to 25 kilovolts. The frequency preferably is 20 Hz. The signal generator controller 44 can be adapted to control the signal generator 40 through a range of frequencies, voltages and at low current (i.e. 100 miliamps) as appropriate for the applications described below.

The present invention relies on the principle of electrical treatment prior to the lancing of the skin to accomplish at least one of pain masking and bodily fluid or blood engorgement.

In one aspect, the electrical pulses stimulate the peripheral terminals of sensory neurons in the body, which cause the release of bioactive substances. These substances for the most part are neuropeptides; substance P and calcitonian gene related peptide. They in turn act on target cells in the periphery of the applied area such as masked cells, immune cells and smooth muscle producing inflammation. This is characterized by redness and warmth, an indication of vasodialation. This phenomenon is known as neurogenetic inflammation.

It has been determined that application of electrical stimulus for a period of approximately 30 seconds will produce vasodialation. Accordingly, after the application of the electrical energy, the lancet 28 is actuated to pierce the skin and produce a quantity of blood which is enhanced by the pretreatment of the surface to produce vasodilatation. In tests outlined in table 1, there is as shown a 77% increase in average blood volume and a 16% increase in the success rate to obtain 0.75 microliters of a sample. For this test, the voltage level was 16 kilovolts at 20 Hz. It should be apparent to those skilled in the art that the electrical parameters set forth in this description are for illustration purposes only based on current investigations and are not to be construed or interpreted as in any way limiting the range of electrical parameters applied within the scope of the present invention.

TABLE 1

Blood Collected (μL)

| | W/O | With |
|---|---|---|
| Test | Stimulation | Stimulation |
| Avg. (μL) | 0.82 | 1.45 |
| Median (μL) | 0.91 | 1.36 |
| StDev | 0.50 | 0.69 |
| Success Rate | 67% | 83% |
| at 0.75 μL | | |

The success rate can further be enhanced by using an expression cap shown in FIG. 2 to permit mechanical compression of the skin site subsequent to lancing. The cap 14 has a plurality of electrode pairs 30 and 32 on a skin contacting face 52 in an array around central opening 34 through which the lancet 28 extends when it is actuated. As shown in FIG. 2, skin contacting face 52 is curved in a negative sigmoid shape with an annular concave section 56 leading from opening 34 to an annular convex section 58. The purpose of this configuration is to allow application of the skin contacting face 52 to the skin site that has been lanced to force bodily fluids such as blood to the incision point in sufficient quantity to obtain a sample for blood analysis.

In order for electrical stimulation to be used to mask pain, the electrical energy is applied for a longer duration prior to making the incision on the skin. This electrical power can be used through the same electrode pairs shown in FIGS. 1 and 2 or it may be as embodied in the device of FIG. 3 having a flat faced skin contacting cap 60 with a plurality of electrode pairs 62 and 64 positioned to generally surround a central opening 66 for the lancing device. The head 60 is connected to a housing 68 containing the elements shown in schematic fashion in FIG. 1. As is the case with FIGS. 1 and 2, the electrode pairs 62, 64 may be oriented in random, circumferential, or radial fashion.

Using either array, the electrodes deliver electrical stimulation to the area to be lanced. This electrical stimulation, depending upon its nature and character, stimulates the sensory neutrons which manipulate the transmission of signals of afferent information to the spinal cord. Electrical stimulation can target the A-delta and C-fibers which deplete neuropeptides content in the terminal ends of the fibers or target the A-beta fiber causing an abundance of signals to be released. The type of outcome is dependent upon the type and intensity of the electrical stimulus such as pulse rate and duration of applied stimulus. This prevents the neuron's ability to transfer information to the central nervous system with respect to trauma or pain to tissues. The stimulus may also target A-beta fibers, which causes an abundance of neuropeptides being released. A-beta fibers are associated with the detection of pressure. As the lancing occurs, the signals transmitted by the A-delta and C-fibers are clouded by the abundance of A-beta fiber signals. This phenomenon tends to reduce the sensitivity of neociceptive pain. To insure adaptability to as many users as possible because of different individual stimulation thresholds, the device is adjustable for the intensity and pulse rate.

Table 2 shows the pain rating with and without electrical stimulation. The electrical stimulation was at 20 Hz for at least 60 seconds prior to lancing. A reduction and/or increase in tolerance of pain were achieved with electrical stimulation. It should be apparent to those skilled in the art that the parameters set forth in this description are for illustration purposes only based on current investigation and are not to be construed or interpreted as in any way limiting the range of electrical parameters applied within the scope of present invention.

TABLE 2

Electrical Stimulus - Pain Rating

| Site | Parameter | Without | With |
|---|---|---|---|
| Forearm | 16 kilovolts ac, | 1 | 0 |
| | 20 Hz | | |
| Finger | 20 kilovolts ac, 20 | 2 | 2* |
| | Hz | | |

2*: Pain intensity of 2 but much more tolerable pain

When the device is intended to be used for both pain masking and engorgement of bodily fluids, the electrical stimulation is applied for approximately 60 seconds and above. After 30 seconds the engorgement of the site with blood is achieved and after approximately 60 seconds the pain masking is realized. Once the pain masking is achieved, the lancing device is fired to lance the skin. Subsequent to lancing, the skin contacting surface 52 may be employed to express bodily fluid or blood from the incision for application to a test strip. Alternatively, different forms of lancing devices may be used which extract a sample for delivery to another test device.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

1. Apparatus for obtaining a sample of bodily fluid through the skin, said apparatus comprising:
a housing,
a plurality of electrodes on said housing and positioned to contact a site on the skin,
an electrical signal generator for applying electrical energy to said electrodes, said electrical signal generator supplying electrical energy in sufficient quantity to stimulate the skin at said site to accomplish at least one of pain masking and bodily fluid engorgement at said site, and
a skin-lancing device mounted in said housing for directing a skin-lancing medium against the skin at said site to form an incision therein subsequent to the application of said electrical energy.

2. Apparatus as claimed in claim 1 wherein said electrodes are positioned in an array to surround said site.

3. Apparatus as claimed in claim 2 wherein said skin-lancing medium is directed through the middle of said electrode array into said skin.

4. Apparatus as claimed in claim 1 wherein said electrical signal generator supplies high voltage AC.

5. Apparatus as claimed in claim 4 wherein the quantity of electrical energy is less for engorgement than pain masking.

6. Apparatus as claimed in claim 5 wherein said electrical energy is applied to said site for approximately 30 seconds, to produce bodily fluid engorgement.

7. Apparatus as claimed in claim 6 wherein said electrical power supply is adapted to apply electrical energy for at least one minute for pain masking and engorgement.

8. Apparatus as claimed in claim 4 where in the voltage range of said electrical power supply is from between approximately 10 to 25 kilovolts at low current (i.e. 100 miliamps).

9. Apparatus as claimed in claim 2 wherein said unit has a plurality of electrode pairs in an array surrounding said site.

10. Apparatus as claimed in claim 1 wherein said apparatus is a self-contained unit.

11. Apparatus as claimed in claim 10 wherein said electrical signal generator has an adjustable level of electrical energy.

12. Apparatus as claimed in claim 1 wherein said electrical signal generator is adapted to adjust the level of electrical energy.

13. Apparatus as claimed in claim 1 further comprising a device adjacent said skin lancing device for indicating a bodily fluid parameter

14. Apparatus as claimed in claim 13 further comprising a capillary passage leading from the skin incision to said bodily fluid indicator device.

15. A method for obtaining a sample of bodily fluid through the skin, said method comprising:
applying electrical energy to a sampling site on said skin of sufficient quantity to stimulate the skin at said site to accomplish at least one of pain masking and bodily fluid engorgement at said site, and
subsequently making an incision at said site to remove a sample of bodily fluid.

16. A method as claimed in claim 15 wherein said electrical energy is applied in an array around said site.

17. A method as claimed in claim 15 wherein said electrical energy is applied in the form of high voltage AC.

18. A method as claimed in claim 17 wherein a lower level of electrical energy is applied for engorgement.

19. A method as claimed in claim 18 wherein said electrical energy is applied for approximately 30 seconds for bodily fluid engorgement.

20. A method as claimed in claim 18 wherein said electrical energy is applied for at least 60 seconds for pain masking and engorgement

21. A method as claimed in claim 17 wherein the voltage range is between approximately 15 and 25 kilovolts.

22. A method as claimed in claim 17 wherein the electrical energy is adjustable.

23. A method as claimed in claim 15 comprising the further step of compressing the site subsequent to making an incision for further enhancing bodily fluid expression.
